(19) United States
(12) Patent Application Publication
(10) Pub. No.: US 2002/0187938 A1
(43) Pub. Date: Dec. 12, 2002

(54) Ghrelin antagonists

(76) Inventor: Romano Deghenghi, St. Cergue (CH)

Correspondence Address: WINSTON & STRAWN, PATENT DEPARTMENT, 1400 L STREET, N.W., WASHINGTON, DC, 20005-3502, US

(21) Appl. No.: 09/902,556

(22) Filed: Jul. 10, 2001

Related U.S. Application Data
(60) Provisional application No. 60/220,178, filed on Jul. 24, 2000.

(51) Int. Cl. A61K 38/08; A61K 38/06; C07K 7/06; C07K 5/10

(52) U.S. Cl. 514/16; 514/17; 514/18; 530/328; 530/329; 530/330

(57) ABSTRACT

Novel peptides are disclosed having antagonistic properties to the Growth Hormone releasing peptide known as Ghrelin. The new peptides are useful in decreasing the circulating levels of Growth Hormone in a mammal and have therapeutic value.

### CROSS-REFERENCE TO RELATED APPLICATIONS

This application claims the benefit of provisional application serial No. 60/220,178 filed Jul. 13, 2000.

### TECHNICAL FIELD

The invention relates to new growth hormone antagonists that can be administered to mammals to decrease the level of circulating growth hormone therein.

### BACKGROUND

Ghrelin is a name for a family of related peptides of 27 or 28 amino acids which have been isolated in the stomach (M. Kojima et al., Nature, 402, 656-660, 1999; H. Hosoda et al., J. Biol. Chem., May 8, 2000) by a distinct cell type in rats and humans. It is further characterized by having an essential octanoyl ester attached to a serine residue. Ghrelins are known to be potent releasers of growth hormone (GH) in animals and man.

Synthetic variations of these peptides were investigated to determine whether improvements can be made on them, and the present invention results from that investigation.

### SUMMARY OF THE INVENTION

It has surprisingly been found that novel peptides of the general formula:

Gly-Ser-Ser(Octanoyl)-Phe-A

where A is —OH, NH2, Leu-Ser-Pro-Glu-X or -Ala-Lys-Leu-Gln-Pro-Arg-B where B is —OH or NH₂ decrease, rather than increase the level of circulating GH in mammals, presumably because these peptides antagonize the effect of the ghrelins. For this reason, these peptides are of value in normalizing or reducing elevated levels of growth hormone such as those found in acromegalic patients or in other tumor related overproduction GH.

### DETAILED DESCRIPTION OF THE INVENTION

The instant peptides can be prepared by a number of synthetic methods such as exemplified in “Chemical Approaches to the Synthesis of Peptides and Proteins” by P. Lloyd-Williams et al., CRC Press, New York 1997, and similar works well known to peptide chemists.

These peptides are administered in aqueous solutions subcutaneously at doses of about 1 to 10 mg/kg of body weight by bolus injection or by slow parenteral infusions. Also, these peptides may be administrated intranasally or intrapulmonary or via a sustained release formulation that includes a biodegradable polymer incorporating the peptide, or by other means well known to those of ordinary skill in the art, such as implantable osmotic pumps and the like.

### EXAMPLES

The following examples illustrate the effectiveness of these novel peptides.

### Example 1

By solid phase synthesis the following peptide was prepared:

Gly-Ser-Ser(Octanoyl)-Phe-Leu-Ser-Pro-Glu

Theoretical MW: 948.9 Found 948.9

Solubility in water: 0.7 mg/ml

Purity by HPLC analysis: 97.8%

The peptide was injected subcutaneously in 10-day old rats at a dose of 300 mg/kg along with a solvent control and Ghrelin, and the circulating GH determined at 15 minutes, as described in R. Deghenghi et al., Life Sciences 54, 1321-1328 (1994). The results were as follows:

| Compounds | GH ng/ml |
|---|---|
| Solvent control | 10.11 ± 1.6 |
| Ghrelin (human) | 139.80 ± 15.37 |
| Peptide of Example 1 | 1.40 ± 0.32 |

This demonstrates that the present peptide antagonizes the effect of the ghrelins by reducing GH release to a level that is almost nil and much lower than the solvent control.

### Example 2

By the same method of Example 1, the following tetradecapeptide was prepared:

Gly-Ser-Ser(Octanoyl)-Phe-Leu-Ser-Pro-Glu-Ala-Lys-Leu-Gln-Pro-Arg

| | |
|---|---|
| Theoretical MW: 1642.7 | Found: 1642.7 |
| Solubility in water: 0.9 mg/ml | |

Purity by HPLC analysis: 95.0%

The peptide was administered to rats as described above in Example 1. The results were as follows:

| Compound | GH ng/ml |
|---|---|
| Solvent control | 10.11 ± 1.6 |
| Ghrelin (human) | 140 ± 15 |
| Peptide of Example 2 | 7.00 ± 3.5 |

Again the inventive peptide is seen to antagonize the effect of the ghrelins by significantly reducing GH release to a level that is below that of the control.

### Example 3

By the same method of Example 1, the following peptide was prepared:

Gly-Ser-Ser(Octanoyl)-Phe

Theoretical MW: 522.4 Found: 522.4

Solubility in water: insoluble

Purity by HPLC analysis: 95.6%

The peptide was administered to rats as described above in Example 1. The results were as follows:

| Compound | GH ng/ml |
|---|---|
| Solvent control | 10.1 ± 1.6 |
| Ghrelin (human) | 139.8 ± 15.4 |
| Peptide of Example 3 | 7.7 ± 1.1 |

Yet again the inventive peptide antagonizes the effect of the ghrelins by significantly reducing GH release to a level that is below that of the control.

What is claimed is:

1. A Ghrelin antagonist peptide of the formula:
Gly-Ser-Ser(Octanoyl)-Phe-A
where A is —OH, NH2, Leu-Ser-Pro-Glu-B, or -Ala-Lys-Leu-Gln-Pro-Arg-B,
where B is —OH or NH2, wherein said peptide antagonizes the effect of ghrelins when administered to a mammal.

2. The peptide of claim 1 specifically as
Gly-S er-Ser(Octanoyl)-Phe-Leu-Ser-Pro-Glu.

3. The peptide of claim 1 specifically as:
Gly-Ser-Ser(Octanoyl)-Phe-Leu-Ser-Pro-Glu-Ala-Lys-Leu-Gln-Pro-Arg.

4. The peptide of claim 1 specifically as:
Gly-S er-S er(Octanoyl)-Phe

5. A pharmaceutical composition comprising peptide of claim 1 in the form of a pharmaceutically acceptable salt.

6. The composition of claim 5 which further comprises a carrier.

7. The composition of claim 5 in the form of a sustained release formation or device for parenteral administration.

8. The peptide of claim 5 in the form of a pharmaceutically acceptable intranasal formulation.

9. The peptide of claim 5 in the form of a pharmaceutically acceptable inhalation formulation.

10. A method of normalizing elevated growth hormone levels in a mammal by administering to a mammal in need of such treatment an effective dose of at least one of the peptides of claim 1.

11. The method of claim 10 wherein the peptide is:
Gly-Ser-Ser(Octanoyl)-Phe-Leu-Ser-Pro-Glu.

12. The method of claim 11 wherein the peptide is:
Gly-S er-S er(Octanoyl)-Phe-Leu-Ser-Pro-Glu-Ala-Lys-Leu-Gln-Pro-Arg.

13. The method of claim 12 wherein the peptide is:
Gly-Ser-Ser(Octanoyl)-Phe.

14. The method of claim 10 wherein the peptide is administered as a sustained release formulation or through a device used for parenteral administration.

15. The method of claim 10 wherein the peptide is administered as a pharmaceutically acceptable intranasal formulation.

16. The method of claim 10 wherein the peptide is administered in a pharmaceutically acceptable inhalation formulation.

17. The method of claim 10 wherein the peptide is administered at a dosage of between about 1 and 10 mg/kg of body weight of the mammal.

18. The method of claim 10 wherein the peptide is administered to a mammal that is acromegalic.
